(19) United States
(12) Patent Application Publication
(10) Pub. No.: US 2026/0108159 A1
(43) Pub. Date: Apr. 23, 2026

(54) Identifying Changes in Vascular Structure Based on Features from PPG Signals

(71) Applicant: Samsung Electronics Co., Ltd., Suwon-si (KR)

(72) Inventors: Michael Freedman, San Francisco, CA (US); Calum MacRae, Wellesley Hills, MA (US); Viswam Nathan, Clovis, CA (US)

(21) Appl. No.: 19/361,492

(22) Filed: Oct. 17, 2025

Related U.S. Application Data
(60) Provisional application No. 63/709,356, filed on Oct. 18, 2024.

(51) Int. Cl. A61B 5/02; A61B 5/00; A61B 5/024; A61B 5/366

(52) U.S. Cl. CPC A61B 5/02007; A61B 5/02416; A61B 5/02438; A61B 5/366; A61B 5/7246; A61B 5/742

(57) ABSTRACT

In one embodiment, a method includes acquiring, by a PPG sensor of a wearable device, photoplethysmography (PPG) signals from a wearer of the wearable device and comparing the acquired PPG signals to a template PPG signal representing a healthy vascular structure. The method further includes determining, based on the comparison, one or more differences between the template PPG signal and the acquired PPG signals; and determining, based on the one or more differences between the template PPG signal and the acquired PPG signals, a current health status of the wearer's vascular structure.

### PRIORITY CLAIM

This application claims the benefit under 35 U.S.C. § 119 of U.S. Provisional Patent Application No. 63/709,356 filed Oct. 18, 2024, which is incorporated by reference herein.

### TECHNICAL FIELD

This application generally relates to identifying changes in vascular anatomy based on features from photoplethysmography (PPG) signals.

### BACKGROUND

Vascular-related health conditions are serious and relatively common diseases. For example, aortic valvular disease occurs where the aortic valve, which controls blood flow from the heart's left ventricle to the rest of the body, does not operate correctly. For example, aortic valve disease can include aortic valve stenosis (narrowing) or aortic valve regurgitation (a leak). Untreated aortic valve disease can lead to progressive health problems including heart failure, arrhythmias, and blood pressure issues. An aortic-valve-disease diagnosis typically requires a cardiologist to order an echocardiogram, for example because the disease has progressed to the point that symptoms are present. Aortic valve problems are often underdiagnosed and diagnosed late, leading to worse disease, in-part because an echocardiogram is not recommended for universal screening, and must instead be ordered by a physician.

### BRIEF DESCRIPTION OF THE DRAWINGS

FIG. 1 illustrates an example method for determining a current health status of a person's vascular structure based on PPG signals obtained from a PPG sensor of a wearable device worn by the person.

FIG. 2 illustrates an example set of PPG signals, where each PPG waveform contains a trough and a peak.

FIG. 3 illustrates other pressure-related vascular conditions that can propagate down a person's vasculature and be indicated by a PPG sensor of a wearable device.

FIG. 4 illustrates an example of a computing system.

### DESCRIPTION OF EXAMPLE EMBODIMENTS

Photoplethysmography (PPG) is a medical technique that uses light to measure blood volume changes in the skin. PPG sensors can be incorporated into a wearable device, such as a ring, a wrist-worn device such as a wristband or smartwatch, a necklace, an earbud, a pair of glasses, etc., and these PPG sensors provide time-series data from peripheral color changes in the capillary bed of an extremity. The morphology of this data is dependent on the character of blood flow out of the aortic valve, and on the vascular structure more generally.

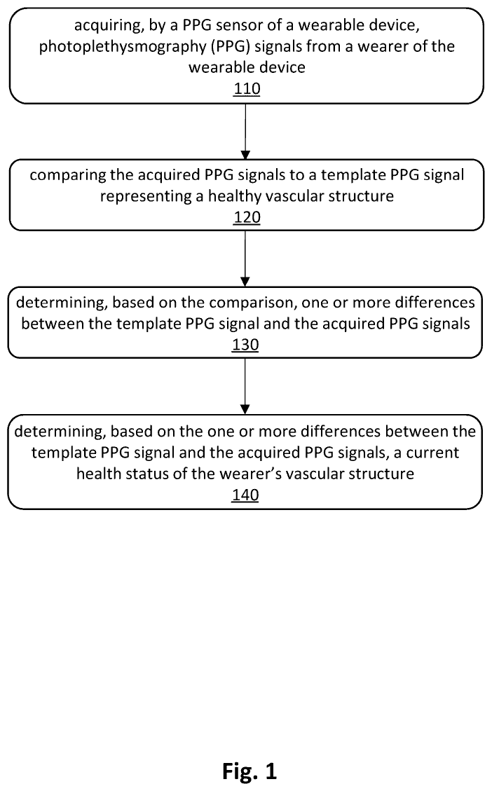

FIG. 1 illustrates an example method for determining a current health status of a person's vascular structure based on PPG signals obtained from a PPG sensor of a wearable device worn by the person. Step 110 of the example method of FIG. 1 includes acquiring, by a PPG sensor of a wearable device, photoplethysmography (PPG) signals from a wearer of the wearable device. The wearable device can be any suitable wearable device, such as for example a ring, a wrist-worn device such as a wristband or smartwatch, a necklace, an earbud, a pair of glasses, etc. The PPG signal may be acquired periodically, such as every hour, every day, every week, etc., although this disclosure contemplates that other acquisition frequencies may be used. A data acquisition episode of step 110 may include obtaining PPG signals for, e.g., a minute, and the PPG signals over that minute may be processed (e.g., to remove noise and outlier signals, and to ensure that the PPG signals are of sufficient quality) and then averaged to arrive at a composite PPG signal over that period of time. This disclosure contemplates that data acquisition periods other than a minute may be used, as well.

Step 120 of the example method of FIG. 1 includes comparing the acquired PPG signals to a template PPG signal representing a healthy vascular structure. In particular embodiments, the template may be a PPG signal (or composite PPG signal derived from multiple PPG signals) that is obtained from that same wearer, but at a previous time when the wearer is determined to have (or likely to have) healthy vasculature. For example, particular embodiments may determine the presence of a chronic aortic valve condition such as aortic stenosis. A PPG signal may be obtained from the wearer, e.g., when the user is relatively young or healthy. This PPG signal may be obtained by a wearable device (e.g., by the same wearable device as performs step 110, or by a different wearable device) or by, e.g., a clinician, and this template-defining PPG signal may be obtained days, months, or years in advance of step 110. For the purposes of creating the template, after obtaining the PPG signal from the wearer, then the PPG signal may be processed (e.g., to remove outliers, improve signal quality, etc.) until a template PPG signal is obtained, e.g., based on the presence of well-defined PPG signal markers for PPG waveforms. This template PPG signal is then stored for the wearer for future comparison, i.e., as performed in step 120.

In particular embodiments, a template may be obtained a relatively long time prior to step 110, and the comparison in step 120 may be performed relatively infrequently. For example, certain chronic aortic valve conditions may occur relatively slowly (e.g., over many years), and therefore a template PPG signal may be obtained many years prior to step 110, and the comparison step 120 may occur every, e.g., month, quarter, year, etc., although other time periods may be used. In other embodiments, certain aortic valve conditions (e.g., a clot that dislodges) may occur relatively quickly (e.g., over a few minutes), and therefore a template signal may be obtained relatively recently (e.g., in the past several minutes, hours, or days etc.) or relatively farther back from step 110. In addition, the comparison of step 120 may occur more frequently when detecting for acute vasculature issues, e.g., step 120 may occur every minute, 30 minutes, hour, day, etc., although this disclosure contemplates that other time frames may be used. As illustrated in the examples above, a template signal and the frequency of comparison step 120 may depend on the particular vascular condition being considered (e.g., whether the condition is a chronic or acute health condition, etc.).

In particular embodiments, a template PPG signal may not come directly from the wearer, but instead may be a PPG signal derived from a set of healthy persons that have one or more demographic conditions in common with the wearer. For example, PPG signals may be obtained from a healthy population of persons having the same age, sex, cardiac risk profile, or other demographic characteristics as the wearer. These PPG signals may be used to create several templates or to create a composite template PPG signal that represents a healthy vascular system for a person having the wearer's demographics. Step 120 may then include comparing this population-derived template with the PPG signals obtained specifically from the wearer in step 110.

Particular embodiments may use dynamic time warping to perform the comparison of step 120. For example, dynamic time warping compares the “distance” between 2 signals while accounting for time differences in which those signals occur. For example, a PPG waveform may be somewhat more compressed than a template (e.g., because the wearer has a somewhat higher heart rate than when the template was acquired), but the overall PPG features may otherwise be the same, and dynamic time warping takes into account this type of temporal-based signal elongation or compression.

Some or all of step 120 may be performed by the wearable device, and/or by another computing device such as a client device (e.g., a smartphone, a personal computer, a tablet, etc.) or a server device, which may access the template PPG signal and the PPG data acquired in step 110 from the wearable device or from another computing device (e.g., from the memory of a client device or a server device).

Step 130 of the example method of FIG. 1 includes determining, based on the comparison, one or more differences between the template PPG signal and the acquired PPG signals. Then, step 140 of the example method of FIG. 1 includes determining, based on the one or more differences between the template PPG signal and the acquired PPG signals, a current health status of the wearer's vascular structure. The differences between the template PPG signal and the acquired PPG signals can indicate the presence of detrimental conditions in the vascular structure.

For instance, one of the key features that presents when stenotic aortic problem are present is that it takes longer for the upstroke to happen during a heartbeat. As a result, the time frame between a person's diastolic blood pressure (i.e., when blood pressure is lowest) to the person's systolic blood pressure (when blood pressure is highest) takes longer within a given cardiac cycle when the person's aortic valve is stenotic and narrow. The opposite effect occurs when the aortic valve is leaky; in other words, the time between diastolic and systolic blood pressure decreases relative to that in a healthy aortic valve. These blood-pressure-related signals propagate down the person's arterial tree and can be detected in the capillary bed by a PPG sensor.

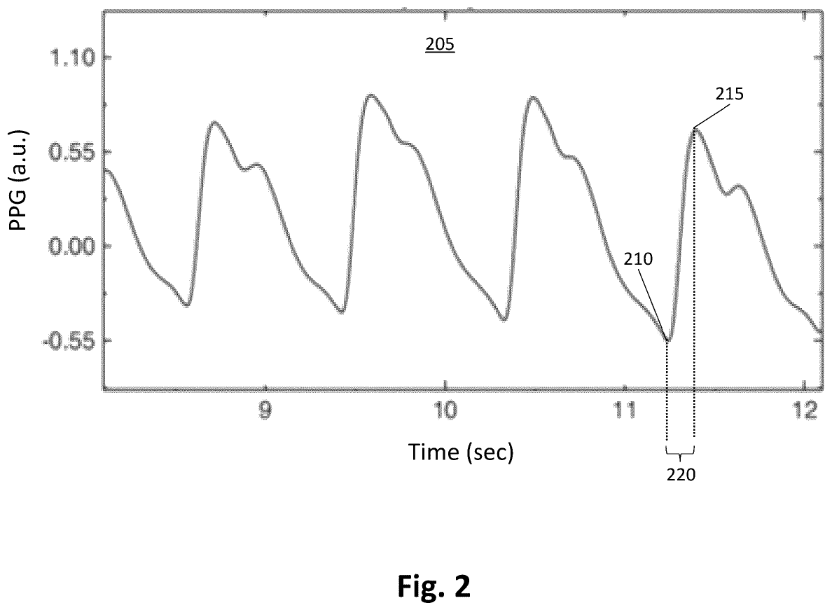

In summary, aortic insufficiency is characterized by a bounding pulse with wide pulse pressure, which presents as corresponding differences in peripheral PPG signal. Similarly, aortic stenosis is characterized by slow ejection of blood through a narrowed aortic valve, resulting in slow upsweep of the PPG signal. Thus, the presence of aortic conditions can be detected by comparing the template PPG signal to the acquired PPG signals and looking at, e.g., the time-to-peak of the upstroke evidenced by the PPG signal. FIG. 2 illustrates an example set of PPG signals 205, where each PPG waveform contains a trough 210 and a peak 215. The time-to-peak 220 characterizes the time of the upstroke, and thus differences in the time-to-peak between template PPG signals and acquired PPG signals can indicate the presence of an aortic condition, as well as indicate the kind of condition that may be present.

In addition, the height of peak 215 relative to trough 210 can indicate a stenotic valve, as a narrower (stenotic) valve blunts the pressure response, resulting in a lower trough-to-peak pressure difference relative to a healthy valve. Here, the presence of trough-to-peak pressure differences may depend on where the PPG sensor is located, as a sensor relatively far from the aortic valve may not evidence this height-related response relative to a sensor that is more closely located to the valve.

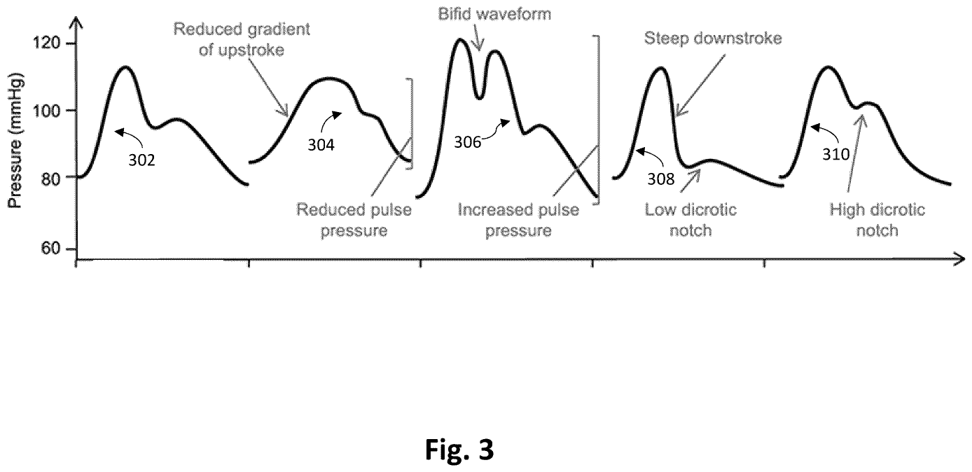

FIG. 3 illustrates other pressure-related conditions that can propagate down a person's vasculature and therefore be indicated by a PPG sensor of a wearable device, such as the presence of progressive dampening of the dicrotic notch as evidenced by the PPG signal. In the example of FIG. 3, waveform 302 is indicative of normal vasculature, while waveform 304 is indicative of aortic stenosis. Waveform 306 is indicative of aortic regurgitation, and waveform 308 is indicative of low systemic vascular resistance (SVR), while waveform 310 is indicative of high SVR. The presence of the indicated features relative to a template PPG signal can therefore indicate the presence of, and even type of, health-related vascular condition.

In particular embodiments, a trained machine-learning model may be used to classify whether the difference between the template PPG signal and the acquired PPG signals are indicative of a condition in the person's vascular structure. In particular embodiments, the trained ML model may also output a classification of the type of condition, e.g., stenosis. Other embodiments may calculate a metric such as a z-score associated with differences in features (e.g., time-to-peak) between the template PPG signal and the acquired PPG signals.

In particular embodiments, the method of FIG. 1 may include surfacing output to the person, either on the wearable device or on another device such as a mobile phone, etc. For example, if the result of step 140 indicates that the person has a health problem with their vascular structure (e.g., a stenotic valve), then the system may surface a message on the UI or play a sound (e.g., a verbal message) alerting the user to the presence of the condition. In particular embodiments, the output may be a recommendation, such as a recommendation that the person consult a health professional. In other embodiments, the output may be (or may include) a specific description of the kind of detected condition (e.g., that the person may have leaky valve). Particular embodiments may provide a higher-level output to the person and may log more detailed data regarding the detected condition (e.g., z-scores, etc.), which may then be accessed and viewed by a medical professional as part of diagnosing the wearer. In particular embodiments, a device (e.g., the wearable device or another device) may alert another person (e.g., an emergency contact, emergency services, etc.) if the result of step 140 detects a severe medical condition, such as a dislodged clot.

Early detection of vascular conditions (e.g., aortic stenosis) can lead to closer monitoring of disease progression and early medical management (e.g. screening for high cholesterol and starting cholesterol-lowering medication), thereby resulting in improved outcomes and decreased disease progression.

Some or all of steps 130 and 140 may be performed by the wearable device or by another device, such as a client device (e.g., a smartphone, a personal computer, a tablet, etc.) or a server device that accesses the underlying data, e.g., from the wearable device or from another intermediary device.

In particular embodiments, one or more additional sensor modalities such as an electrocardiogram (ECG) sensor or a ballistocardiogram (BCG) sensor (e.g., an accelerometer) may be combined with PPG sensor data to improve detection of vasculature health conditions. For example, both ECG and PPG signals may be obtained by respective sensors of a wearable device. High-voltage QRS patterns in the ECG data may be analyzed to detect, e.g., left ventricular hypertrophy (LVH), which is a condition in which the heart muscle grows in size, resulting in larger cardiac electrical signals. For example, features such as the biggest R or S wave in limb leads (I, II, III) being greater than or equal to 20 mm, a left axis deviation (e.g., QRS axis −30 or more negative), or a QRS duration greater than 0.09 seconds may all be indicative of LVH. The combination of ECG and PPG can then be used to determine the presence of conditions such as aortic stenosis

[symbol]

In particular embodiment, a PPG sensor may be a standard PPG sensor, e.g., that uses emission and absorption of infrared light to detect the PPG signals. In other embodiments, a PPG sensor may use an optical camera that captures images of a region of the person's skin to detect fine color changes in the wearer's skin, from which PPG signals can be estimated.

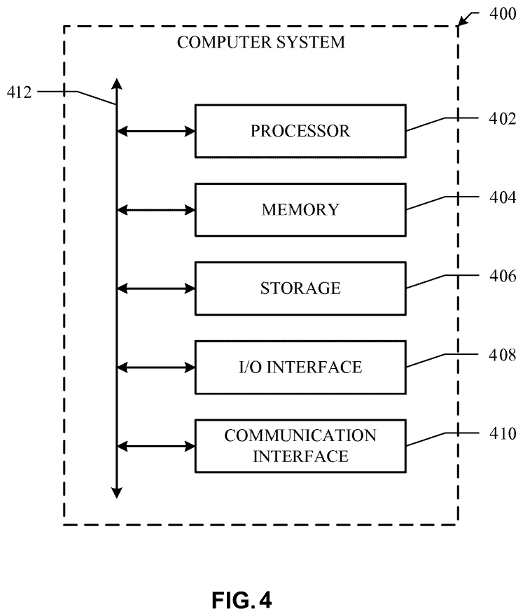

FIG. 4 illustrates an example general-purpose computer system 400. FIG. 4 illustrates the main processors and memory in general-purpose computer system 400 and not the secured hardware portions described above. In particular embodiments, one or more computer systems 400 perform one or more steps of one or more methods described or illustrated herein. In particular embodiments, one or more computer systems 400 provide functionality described or illustrated herein. In particular embodiments, software running on one or more computer systems 400 performs one or more steps of one or more methods described or illustrated herein or provides functionality described or illustrated herein. Particular embodiments include one or more portions of one or more computer systems 400. Herein, reference to a computer system may encompass a computing device, and vice versa, where appropriate. Moreover, reference to a computer system may encompass one or more computer systems, where appropriate.

This disclosure contemplates any suitable number of computer systems 400. This disclosure contemplates computer system 400 taking any suitable physical form. As example and not by way of limitation, computer system 400 may be an embedded computer system, a system-on-chip (SOC), a single-board computer system (SBC) (such as, for example, a computer-on-module (COM) or system-on-module (SOM)), a desktop computer system, a laptop or notebook computer system, an interactive kiosk, a mainframe, a mesh of computer systems, a mobile telephone, a personal digital assistant (PDA), a server, a tablet computer system, or a combination of two or more of these. Where appropriate, computer system 400 may include one or more computer systems 400; be unitary or distributed; span multiple locations; span multiple machines; span multiple data centers; or reside in a cloud, which may include one or more cloud components in one or more networks. Where appropriate, one or more computer systems 400 may perform without substantial spatial or temporal limitation one or more steps of one or more methods described or illustrated herein. As an example and not by way of limitation, one or more computer systems 400 may perform in real time or in batch mode one or more steps of one or more methods described or illustrated herein. One or more computer systems 400 may perform at different times or at different locations one or more steps of one or more methods described or illustrated herein, where appropriate.

In particular embodiments, computer system 400 includes a processor 402, memory 404, storage 406, an input/output (I/O) interface 408, a communication interface 410, and a bus 412. Although this disclosure describes and illustrates a particular computer system having a particular number of particular components in a particular arrangement, this disclosure contemplates any suitable computer system having any suitable number of any suitable components in any suitable arrangement.

In particular embodiments, processor 402 includes hardware for executing instructions, such as those making up a computer program. As an example and not by way of limitation, to execute instructions, processor 402 may retrieve (or fetch) the instructions from an internal register, an internal cache, memory 404, or storage 406; decode and execute them; and then write one or more results to an internal register, an internal cache, memory 404, or storage 406. In particular embodiments, processor 402 may include one or more internal caches for data, instructions, or addresses. This disclosure contemplates processor 402 including any suitable number of any suitable internal caches, where appropriate. As an example and not by way of limitation, processor 402 may include one or more instruction caches, one or more data caches, and one or more translation lookaside buffers (TLBs). Instructions in the instruction caches may be copies of instructions in memory 404 or storage 406, and the instruction caches may speed up retrieval of those instructions by processor 402. Data in the data caches may be copies of data in memory 404 or storage 406 for instructions executing at processor 402 to operate on; the results of previous instructions executed at processor 402 for access by subsequent instructions executing at processor 402 or for writing to memory 404 or storage 406; or other suitable data. The data caches may speed up read or write operations by processor 402. The TLBs may speed up virtual-address translation for processor 402. In particular embodiments, processor 402 may include one or more internal registers for data, instructions, or addresses. This disclosure contemplates processor 402 including any suitable number of any suitable internal registers, where appropriate. Where appropriate, processor 402 may include one or more arithmetic logic units (ALUs); be a multi-core processor; or include one or more processors 402. Although this disclosure describes and illustrates a particular processor, this disclosure contemplates any suitable processor.

In particular embodiments, memory 404 includes main memory for storing instructions for processor 402 to execute or data for processor 402 to operate on. As an example and not by way of limitation, computer system 400 may load instructions from storage 406 or another source (such as, for example, another computer system 400) to memory 404. Processor 402 may then load the instructions from memory 404 to an internal register or internal cache. To execute the instructions, processor 402 may retrieve the instructions from the internal register or internal cache and decode them. During or after execution of the instructions, processor 402 may write one or more results (which may be intermediate or final results) to the internal register or internal cache. Processor 402 may then write one or more of those results to memory 404. In particular embodiments, processor 402 executes only instructions in one or more internal registers or internal caches or in memory 404 (as opposed to storage 406 or elsewhere) and operates only on data in one or more internal registers or internal caches or in memory 404 (as opposed to storage 406 or elsewhere). One or more memory buses (which may each include an address bus and a data bus) may couple processor 402 to memory 404. Bus 412 may include one or more memory buses, as described below. In particular embodiments, one or more memory management units (MMUs) reside between processor 402 and memory 404 and facilitate accesses to memory 404 requested by processor 402. In particular embodiments, memory 404 includes random access memory (RAM). This RAM may be volatile memory, where appropriate Where appropriate, this RAM may be dynamic RAM (DRAM) or static RAM (SRAM). Moreover, where appropriate, this RAM may be single-ported or multi-ported RAM. This disclosure contemplates any suitable RAM. Memory 404 may include one or more memories 404, where appropriate. Although this disclosure describes and illustrates particular memory, this disclosure contemplates any suitable memory.

In particular embodiments, storage 406 includes mass storage for data or instructions. As an example and not by way of limitation, storage 406 may include a hard disk drive (HDD), a floppy disk drive, flash memory, an optical disc, a magneto-optical disc, magnetic tape, or a Universal Serial Bus (USB) drive or a combination of two or more of these. Storage 406 may include removable or non-removable (or fixed) media, where appropriate. Storage 406 may be internal or external to computer system 400, where appropriate. In particular embodiments, storage 406 is non-volatile, solid-state memory. In particular embodiments, storage 406 includes read-only memory (ROM). Where appropriate, this ROM may be mask-programmed ROM, programmable ROM (PROM), erasable PROM (EPROM), electrically erasable PROM (EEPROM), electrically alterable ROM (EAROM), or flash memory or a combination of two or more of these. This disclosure contemplates mass storage 406 taking any suitable physical form. Storage 406 may include one or more storage control units facilitating communication between processor 402 and storage 406, where appropriate. Where appropriate, storage 406 may include one or more storages 406. Although this disclosure describes and illustrates particular storage, this disclosure contemplates any suitable storage.

In particular embodiments, I/O interface 408 includes hardware, software, or both, providing one or more interfaces for communication between computer system 400 and one or more I/O devices. Computer system 400 may include one or more of these I/O devices, where appropriate. One or more of these I/O devices may enable communication between a person and computer system 400. As an example and not by way of limitation, an I/O device may include a keyboard, keypad, microphone, monitor, mouse, printer, scanner, speaker, still camera, stylus, tablet, touch screen, trackball, video camera, another suitable I/O device or a combination of two or more of these. An I/O device may include one or more sensors. This disclosure contemplates any suitable I/O devices and any suitable I/O interfaces 408 for them. Where appropriate, I/O interface 408 may include one or more device or software drivers enabling processor 402 to drive one or more of these I/O devices. I/O interface 408 may include one or more I/O interfaces 408, where appropriate. Although this disclosure describes and illustrates a particular I/O interface, this disclosure contemplates any suitable I/O interface.

In particular embodiments, communication interface 410 includes hardware, software, or both providing one or more interfaces for communication (such as, for example, packet-based communication) between computer system 400 and one or more other computer systems 400 or one or more networks. As an example and not by way of limitation, communication interface 410 may include a network interface controller (NIC) or network adapter for communicating with an Ethernet or other wire-based network or a wireless NIC (WNIC) or wireless adapter for communicating with a wireless network, such as a WI-FI network. This disclosure contemplates any suitable network and any suitable communication interface 410 for it. As an example and not by way of limitation, computer system 400 may communicate with an ad hoc network, a personal area network (PAN), a local area network (LAN), a wide area network (WAN), a metropolitan area network (MAN), or one or more portions of the Internet or a combination of two or more of these. One or more portions of one or more of these networks may be wired or wireless. As an example, computer system 400 may communicate with a wireless PAN (WPAN) (such as, for example, a BLUETOOTH WPAN), a WI-FI network, a WI-MAX network, a cellular telephone network (such as, for example, a Global System for Mobile Communications (GSM) network), or other suitable wireless network or a combination of two or more of these. Computer system 400 may include any suitable communication interface 410 for any of these networks, where appropriate. Communication interface 410 may include one or more communication interfaces 410, where appropriate. Although this disclosure describes and illustrates a particular communication interface, this disclosure contemplates any suitable communication interface.

In particular embodiments, bus 412 includes hardware, software, or both coupling components of computer system 400 to each other. As an example and not by way of limitation, bus 412 may include an Accelerated Graphics Port (AGP) or other graphics bus, an Enhanced Industry Standard Architecture (EISA) bus, a front-side bus (FSB), a HYPERTRANSPORT (HT) interconnect, an Industry Standard Architecture (ISA) bus, an INFINIBAND interconnect, a low-pin-count (LPC) bus, a memory bus, a Micro Channel Architecture (MCA) bus, a Peripheral Component Interconnect (PCI) bus, a PCI-Express (PCIe) bus, a serial advanced technology attachment (SATA) bus, a Video Electronics Standards Association local (VLB) bus, or another suitable bus or a combination of two or more of these. Bus 412 may include one or more buses 412, where appropriate. Although this disclosure describes and illustrates a particular bus, this disclosure contemplates any suitable bus or interconnect.

Herein, a computer-readable non-transitory storage medium or media may include one or more semiconductor-based or other integrated circuits (ICs) (such, as for example, field-programmable gate arrays (FPGAs) or application-specific ICs (ASICs)), hard disk drives (HDDs), hybrid hard drives (HHDs), optical discs, optical disc drives (ODDs), magneto-optical discs, magneto-optical drives, floppy diskettes, floppy disk drives (FDDs), magnetic tapes, solid-state drives (SSDs), RAM-drives, SECURE DIGITAL cards or drives, any other suitable computer-readable non-transitory storage media, or any suitable combination of two or more of these, where appropriate. A computer-readable non-transitory storage medium may be volatile, non-volatile, or a combination of volatile and non-volatile, where appropriate.

Herein, “or” is inclusive and not exclusive, unless expressly indicated otherwise or indicated otherwise by context. Therefore, herein, “A or B” means “A, B, or both,” unless expressly indicated otherwise or indicated otherwise by context. Moreover, “and” is both joint and several, unless expressly indicated otherwise or indicated otherwise by context. Therefore, herein, “A and B” means “A and B, jointly or severally,” unless expressly indicated otherwise or indicated otherwise by context.

This disclosure contemplates a system that includes one or more non-transitory computer readable storage media storing instructions; and one or more processors coupled to the one or more non-transitory computer readable storage media and operable to execute the instructions to perform certain functions includes embodiments in which those functions are performed by a single processor, embodiments in which those functions are performed by multiple processors that each perform all the functions, and embodiments in which those functions are performed by multiple processors (e.g., in separate computing devices) where each processor performs at least one function but less than all recited functions.

The scope of this disclosure encompasses all changes, substitutions, variations, alterations, and modifications to the example embodiments described or illustrated herein that a person having ordinary skill in the art would comprehend. The scope of this disclosure is not limited to the example embodiments described or illustrated herein. Moreover, although this disclosure describes and illustrates respective embodiments herein as including particular components, elements, feature, functions, operations, or steps, any of these embodiments may include any combination or permutation of any of the components, elements, features, functions, operations, or steps described or illustrated anywhere herein that a person having ordinary skill in the art would comprehend.

What is claimed is:

1. A method comprising:
acquiring, by a PPG sensor of a wearable device, photoplethysmography (PPG) signals from a wearer of the wearable device;
comparing the acquired PPG signals to a template PPG signal representing a healthy vascular structure;
determining, based on the comparison, one or more differences between the template PPG signal and the acquired PPG signals; and
determining, based on the one or more differences between the template PPG signal and the acquired PPG signals, a current health status of the wearer's vascular structure.

2. The method of claim 1, wherein the template PPG signal represents a healthy aortic valve and determining the current health status of the wearer's vascular structure comprises determining a current health status of the wearer's aortic valve.

3. The method of claim 2, wherein the current health status of the wearer's aortic valve comprises a likelihood that the wearer has an aortic valvular disease.

4. The method of claim 3, wherein the one or more differences comprise a difference in a time to peak between the template PPG signal and one or more PPG waveforms in the acquired PPG signals.

5. The method of claim 3, wherein the one or more differences comprise a dampening in a dicrotic notch in the acquired PPG signals compared to the template PPG signal.

6. The method of claim 1, further comprising providing for display on a display of an electronic computing device, a user interface representing the current health status of the wearer's vasculature structure.

7. The method of claim 1, wherein the template PPG signal is based on a previous PPG signal of the wearer.

8. The method of claim 1, wherein the template PPG signal is based on a plurality of previous PPG signals from a plurality of persons having a healthy vasculature structure.

9. The method of claim 8, wherein the plurality of persons are defined based on a similarity between one or more demographic characteristics of the wearer and corresponding one or more demographic characteristics of the plurality of persons.

10. The method of claim 1, further comprising:
acquiring, by an electrocardiogram (ECG) sensor of the wearable device, ECG signals from the wearer of the wearable device; and
determining the current health status of the wearer's vascular structure based on (1) the acquired ECG signals and (2) the one or more differences between the template PPG signal and the acquired PPG signals.

11. The method of claim 10, further comprising analyzing high-voltage QRS patterns in the ECG signal to detect a left ventricular hypertrophy (LVH) of the wearer, wherein the current health status of the wearer comprises an aortic stenosis of the wearer.

12. A system comprising:
a wearable device comprising a photoplethysmography (PPG) sensor configured to acquire PPG signals from a wearer of the wearable device; and
one or more non-transitory computer readable storage media storing instructions, and one or more processors coupled to the one or more non-transitory computer readable storage media and operable to execute the instructions to:
compare the acquired PPG signals to a template PPG signal representing a healthy vascular structure;
determine, based on the comparison, one or more differences between the template PPG signal and the acquired PPG signals; and
determine, based on the one or more differences between the template PPG signal and the acquired PPG signals, a current health status of the wearer's vascular structure.

13. The system of claim 12, wherein the template PPG signal represents a healthy aortic valve and determining the current health status of the wearer's vascular structure comprises determining a current health status of the wearer's aortic valve.

14. The system of claim 13, wherein the current health status of the wearer's aortic valve comprises a likelihood that the wearer has an aortic valvular disease.

15. The system of claim 14, wherein the one or more differences comprise a difference in a time to peak between the template PPG signal and one or more PPG waveforms in the acquired PPG signals.

16. The system of claim 12, further comprising one or more processors that are operable to execute the instructions to provide for display on a display of an electronic computing device, a user interface representing the current health status of the wearer's vasculature structure.

17. The system of claim 12, wherein the template PPG signal is based on a previous PPG signal of the wearer.

18. The system of claim 12, wherein the template PPG signal is based on a plurality of previous PPG signals from a plurality of persons having a healthy vasculature structure.

19. The system of claim 12, wherein the wearable device further comprises an electrocardiogram (ECG) sensor configured to acquire ECG signals from the wearer of the wearable device; and the one or more processors are further configured to execute the instructions to determine the current health status of the wearer's vascular structure based on (1) the acquired ECG signals and (2) the one or more differences between the template PPG signal and the acquired PPG signals.

20. One or more non-transitory computer-readable storage media comprising instructions that are operable when executed by one or more processors to:
acquire, by a PPG sensor of a wearable device, photoplethysmography (PPG) signals from a wearer of the wearable device;
compare the acquired PPG signals to a template PPG signal representing a healthy vascular structure;
determine, based on the comparison, one or more differences between the template PPG signal and the acquired PPG signals; and
determine, based on the one or more differences between the template PPG signal and the acquired PPG signals, a current health status of the wearer's vascular structure.
